(19) United States
(12) Plant Patent Application Publication
(10) Pub. No.: US 2022/0117134 P1
(43) Pub. Date: Apr. 14, 2022

(54) Sansevieria Plant Named 'OSV Sans 006'

(50) ;

(71) Applicant: ForemostCo., Inc., Miami, FL (US)

(72) Inventor: Folkert Hoekstra, Santa Ana (CR)

(21) Appl. No.: 17/495,518

(22) Filed: Oct. 6, 2021

Related U.S. Application Data
(60) Provisional application No. 63/204,558, filed on Oct. 8, 2020.

(51) Int. Cl. A01H 6/32

(52) U.S. Cl. PLT/382

(57) ABSTRACT

A new and distinct Sansevieria plant having intense dark green leaves with a vertical striping pattern and no speckling present.

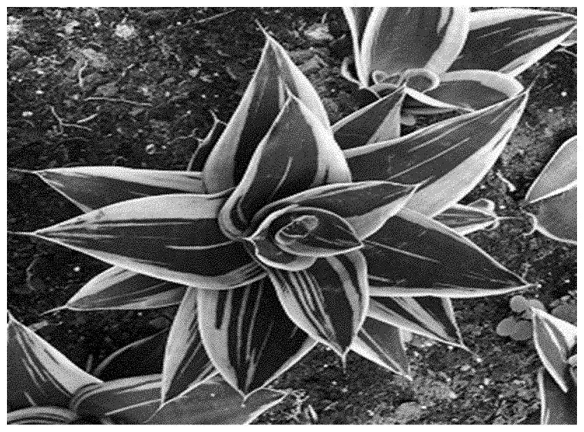

### CROSS REFERENCE TO RELATED APPLICATION(S)

This application claims the benefit of priority from U.S. Provisional Patent Application No. 63/204,558, filed Oct. 8, 2020, which is incorporated herein by reference in its entirety.

Botanical classification: Dracaena sansevieria.

Varietal denomination: ‘OSV Sans 006’.

### BACKGROUND OF THE INVENTION

The present invention comprises a new and distinct variety of Sansevieria plant, botanically known as Dracaena sansevieria, having the varietal name of ‘OSV Sans 006’ and commercially referred to as “Glimmering Star”. ‘OSV Sans 006’ is the result of a cross conducted in Costa Rica in a planned breeding program by the inventor. The new variety was selected and first asexually reproduced by cuttings in Costa Rica. ‘OSV Sans 006’ has been trial and field tested and has been found to retain its distinctive characteristics and remain true to type through successive propagations. The present invention has not been evaluated under all possible environmental conditions. The phenotype may vary with variations in environment without a change in the genotype of the plant.

When compared to Sansevieria variety named ‘OSV Sans 011’ (U.S. Plant patent application Ser. No. ______), the new variety exhibits a similar rosette leaf formation with leaves growing upward and then curving outward that have no speckling or spots present. However, ‘OSV Sans 006’ differs from ‘OSV Sans 011’ in having a vertical striping pattern along its leaves. Further, the following characteristics distinguish ‘OSV Sans 006’ from other Sansevieria varieties known to the breeder:
Rosette leaf formation;
No leaf specking or spots with fluid coloration;
Leaves grow upward and then curve outward; and
Vertical line pattern present along leaves.

### DESCRIPTION OF THE DRAWING

The accompanying photographic drawing taken at approximately 18 weeks of age illustrates the new variety, with the colors being as nearly true as is possible with color illustrations of this type.

### DESCRIPTION OF THE PLANT

The following detailed description sets forth the characteristics of the new variety at approximately 2 years of age. The data which defines these characteristics was collected in a greenhouse in September of 2021 in Winter Garden, Fla. Plants of the new variety were grown under 2,000 foot candles of artificial light in 15 cm pots in a climate-controlled greenhouse in Winter Garden, Fla. having 86% relative humidity and an average temperature of 28° C. Color references are primarily to the Munsell Plant Tissue Color Book, 2019 publication, except where general color terms are used.
Plant:
   Time to initiate roots.—About 12 days at an average of 23° C. night temperatures and 30° C. day temperatures.
   Time to develop roots.—About 21 days at an average of 23° C. night temperatures and 30° C. day temperatures.
   Time to produce a finished plant from a rooted cutting.—About 8 weeks in a 15 cm container.
   Rooting habit and description.—Hair-like roots spread out and wrap at the base.
   Form.—Spreading.
   Height from media surface to top of foliage.—20.3 cm.
   Plant diameter.—22.9 cm.
   Plant shape.—Rosette, having a circular arrangement of the leaves.
   Vigor.—Strong.
   Strength.—Rigid, with no need for artificial support.
   Disease resistance/susceptibility.—Resistant to most Sansevieria diseases.
   Pest resistance/susceptibility.—Resistant to most Sansevieria pests.
   Temperature tolerance.—Sensitive to temperatures below 7° C. or above 38° C.
   Drought tolerance.—Very tolerant due to the ability of the leaves to retain water.
   Flowers.—None observed to date.
   Seeds/fruit.—None present.
   Stem.—Length: 1.5 cm. Diameter: 2.5 cm. Shape: Vertical column. Color: 2.5GY 8/2. Texture: Coriaceous and glabrous. Strength: Sturdy and rigid. Internode length: Not applicable.
   Leaves.—Arrangement: Basal rosette. Average number per plant: 18. Length: 14.7 cm. Width: 6.4 cm. Shape of leaf (generally): Gladiate. Shape of apex: Apiculate. Shape of base: Cuneate. Margin description: Entire. Aspect: Mostly erect, arching outwardly to about 75°. Texture and luster: Upper surface: Coriaceous and glabrous. Lower surface: Coriaceous and glabrous. Pubescence: Upper surface: None present. Lower surface: None present. Fragrance: None present. Color: Young leaves: Upper surface: 5Y 8/4 margins that fade into 2.5GY 6/6; 7.5GY 3/2 covers most of the leaf surface; 5Y 8/4 vertical lines that fade into 2.5GY 6/6 are randomly present. Lower surface: Thin 5Y 8/4 margins; 7.5GY 5/2 covers the remainder of the leaf surface. Mature leaves: Upper surface: 5Y 8/4 margins that fade into 2.5GY 6/6; 7.5GY 3/2 that fades into 7.5GY 4/4 and then 2.5GY 5/6 covers the remainder of the leaf surface. Lower surface: 5Y 8/4 margins; 7.5GY 3/4 that fades into 5GY 6/8 and 2.5GY 8/8 covers the remainder of the leaf surface.
   Veins.—Venation pattern: Parallel. Color: Lower surface: 7.5GY 3/4, 5GY 6/8, and 2.5GY 8/8 that fade into each other.
   Sheath.—Length: 1.3 cm. Width: 3.8 cm. Color: 2.5GY 8/2. Texture: Coriaceous and glabrous.

I claim:

1. A new and distinct variety of Sansevieria plant, as is herein illustrated and described.
